(19) United States
(12) Patent Application Publication
(10) Pub. No.: US 2012/0298112 A1
(43) Pub. Date: Nov. 29, 2012

(54) Endotracheal tube and stylet

(76) Inventor: Larry Paskar, Chesterfield, MO (US)

(21) Appl. No.: 13/134,054

(22) Filed: May 27, 2011

(51) Int. Cl. A61M 16/04

(52) U.S. Cl. 128/207.14

(57) ABSTRACT

An endotracheal tube having more than six degrees of freedom. The tube includes a first element having a distal end portion which is capable of achieving a curved configuration and a second element having a distal end portion which is capable of achieving a curved configuration. The first and second elements are rotationally and translationally connected to each other. The structure may also be used as a stylet for an endotracheal tube.

### CROSS-REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

1. Field of the Invention

The present invention relates to endotracheal tubes and stylets therefor, and more particularly to such tubes and stylets which are especially useful in intubating living beings with abnormal airways.

2. Description of the Prior Art

Tubes for insertion into a patient's airway (and more specifically into the patient's trachea) for the purpose of establishing and maintaining the integrity of the airway and for allowing the exchange of gases to and from the lungs are well known. These tubes are conventionally inserted orally (an orotracheal tube) or nasally (a nasotracheal tube). Tracheostomy tubes are also known. Most endotracheal tubes are formed with a curvature selected to approximate the shape of the airway through which it is to be inserted. The tubes are designed to be sufficiently long enough and sufficiently small enough to pass safely between the vocal cords of the patient into the trachea. The tubes come in various sizes, which the medical practitioner selects among to choose the “right” size for the particular patient. These tubes may or may not have an inflatable cuff near the tube's distal end to provide a seal against the walls of the airway.

Endotracheal tubes may be used both in “routine” and emergency situations. A typical “routine” situation is the placement of the endotracheal tube for purposes of administering anesthetics. Although the amount of time it takes to intubate a patient is important in both situations, it can be critical in the emergency situation. As alluded to above, the medical practitioner typically chooses the “right” size endotracheal tube for the patient. This choice is based on the practitioners assessment of the body size of the patient and usually assumes that the airway for this particular patient is “normal” (i.e., average) for that body size. That assumption is not always correct, and in fact can be grossly in error when the airway has been traumatized or is otherwise malformed. The angle which the trachea makes with the esophagus can vary considerably among individuals, as can practically every portion of the body making up the airway to that point. The oral cavity, the nasal cavity, the tongue, the pharynx and the laryngeal cavity may all have shapes that vary widely from individual to individual.

Failure to intubate properly may result in placement of the endotracheal tube in the esophagus rather than the trachea. In fact, it some cases it is much easier to intubate the esophagus than to intubate the trachea. Particularly in the emergency situation, there may not be sufficient time to correct the mis-placement before the patient expires.

In some instances the intubation is so difficult that an intubation stylet is used with the endotracheal tube to help ensure proper placement. Such stylets typically are bendable and of sufficient rigidity to cause the endotracheal tube to take the overall shape of the stylet as it passes over the stylet. Stylets typically are bent into the desired shape outside the patient's body based upon the medical practitioner's best guess as to the needed shape and then inserted into the body. If the guess was wrong, the stylet in many cases must be removed from the body and re-shaped, which results in undesirable delay.

### OBJECTS OF THE INVENTION

Among the various objects and features of the present invention may be noted the provision of an improved endotracheal tube which facilitates intubation of the patient.

Another object is the provision of such a tube which is much less dependent on the patient having a “normal” airway.

A third object is the provision of such a tube which may be shaped into the required shape during intubation.

Other objects and features will be in part apparent and in part pointed out hereinafter.

### SUMMARY OF THE INVENTION

In a first aspect of the present invention, there is provided an endotracheal tube having more than six degrees of freedom.

In a second aspect of the present invention, the endotracheal tube has a first element with a distal end portion capable of achieving a curved configuration and a second element having a distal end portion capable of achieving a curved configuration, said first and second elements being rotationally and translationally connected to each other.

In a third aspect of the present invention, an endotracheal tube stylet includes a first element having a distal end portion capable of achieving a curved configuration, and a second element having a distal end portion capable of achieving a curved configuration. The first and second elements are disposed with respect to each other to form a stylet having a diameter of less than approximately 8 mm.

### BRIEF DESCRIPTION OF THE DRAWINGS

FIG. 1 is a schematic view of the endotracheal tube of the present invention inserted in a human patient;

FIG. 2 is a cross-sectional view of a distal portion of the endotracheal tube of the present invention;

FIG. 3 is a set of schematic views showing a few of the vast number of shapes achievable by the endotracheal tube of the present invention;

FIG. 4 is an illustration of an alternative structure for the endotracheal tube of the present invention;

FIG. 5 illustrates a fiberscope and pull-wires used in connection with the endotracheal tube of the present invention.

Similar reference characters indicate similar parts throughout the several views of the drawings.

### DETAILED DESCRIPTION

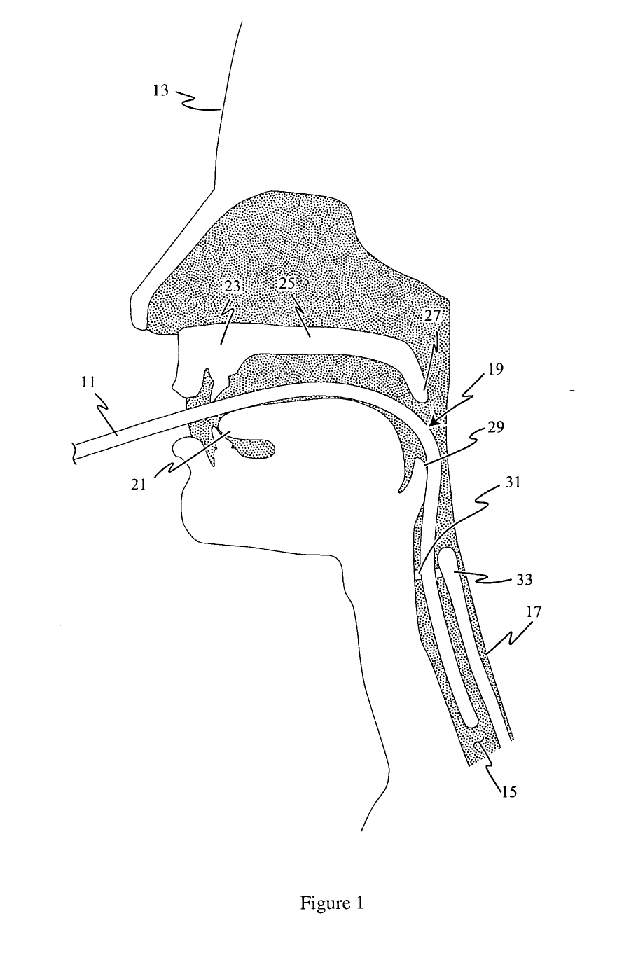

Turning now to the drawings, an endotracheal tube 11 of the present invention is shown correctly placed in a living being (in this case a patient 13). Although tube 11 may include an inflatable cuff as mentioned above, for purposes of clarity the cuff is not shown in FIG. 1. FIG. 1 illustrates perhaps the most favorable configuration of the patient's airway for proper placement of endotracheal tube 11 in the trachea 15 rather than in the esophagus 17. Even with this favorable configuration, note that the degree of curvature of the tube 11 changes significantly at 19 so as to make it possible to enter the trachea. Absent such a radical change in curvature, the intubation of the esophagus 17 instead of the trachea 15 is almost inevitable.

Note that the tongue 21, the hard palate 23, the soft palate 25, the uvula 27, the epiglottis 29, vocal cords 31, and the larynx 33 are all “normal” in shape and almost appear to be cooperating to allow proper placement of endotracheal tube 11. Such a perfect situation is rarely encountered. Instead, any of these components of the airway may be misshapen or injured in some way. Moreover, the diagram of FIG. 1 shows the various components of the airway in two dimensions. In reality, of course, the problem of proper placement of the endotracheal tube 11 is a three-dimensional problem, so mis-alignment of any of the components of the airway can result in an even more difficult intubation.

When using conventional endotracheal tubes, the medical professional often finds it difficult to intubate difficult airways. The conventional endotracheal tube is a relatively rigid device which can be manipulated by the user in only six degrees of freedom. (It is well-known that a rigid body in three dimensions has only six degrees of freedom. “In general, a rigid body in d dimensions has d(d+1)/2 degrees of freedom (d translations and d(d−1)/2 rotations”—see http://en.wikipedia.org/wiki/Degrees_of_freedom_%28mechanics %29 for example.)

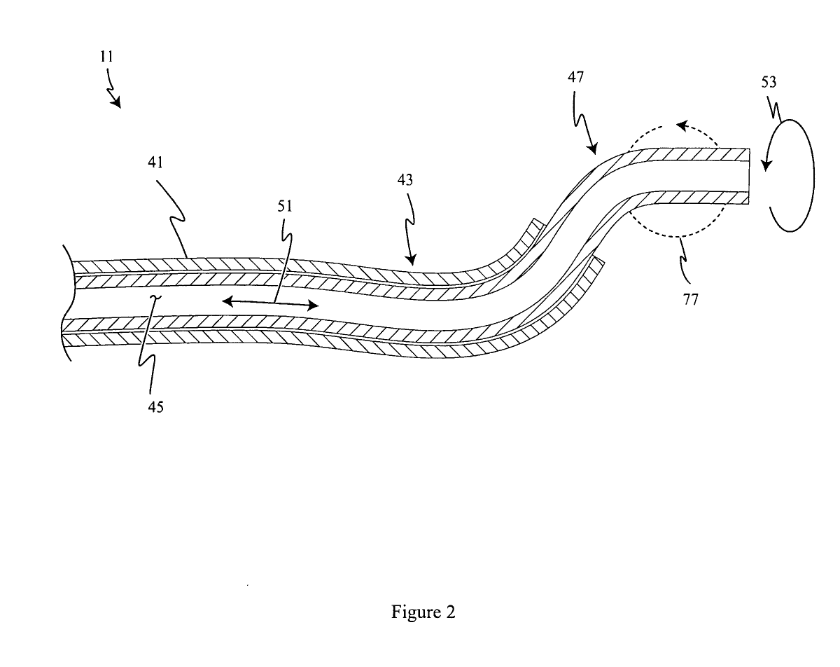

The endotracheal tube 11 of the present invention solves many of the problems outlined above. Endotracheal tube 11 is a composite structure having first and second parts or elements which are movable with respect to each other so that the composite structure has more than six degrees of freedom. For example, one embodiment of tube 11 is shown in FIG. 2 as having a first element or tube 41 with a distal end portion 43 which is capable of achieving a curved configuration and a second element or tube 45 having a distal end portion 47 which is capable of achieving a curved configuration. The first and second elements 41 and 45 are rotationally and translationally connected to each other—that is, the tubes 41 and 45 are connected to each other and are movable translationally (as indicated by arrow 51) and rotationally (as indicated by arrow 53 with respect to each other. Although the curvature in distal end portions 43 and 47 may be preformed and therefore relatively fixed, it is preferred that the curvature of at least one of the portions 43 and/or 47 be controllable by the medical practitioner by a pull-wire or the like (not shown). Pull-wires are well-known in the catheter art. In any event the ability of the medical practitioner to move the curved distal end portions translationally and rotationally with respect to each other results in a vast number of possible shapes that can be formed by the composite endotracheal tube 11, a number of shapes so vast that they can accommodate practically any variation in the endotracheal pathway of the patient 13. In those cases where the degree of curvature of the distal end portions is also controllable by the medical practitioner, the vast number of possible controllable shapes is further multiplied.

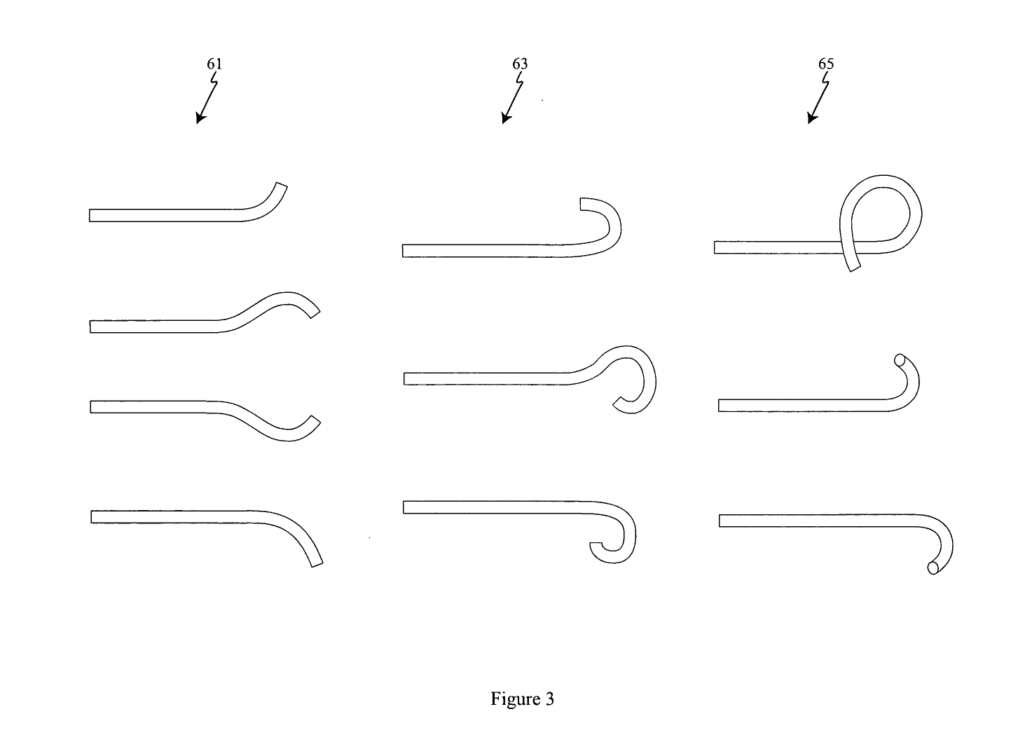

FIG. 3 shows in schematic form a handful of the possible shapes that can be achieved to accommodate variations in the patient's endotracheal pathway. Note that although the first and second column of shapes 61 and 63 are generally planar, out-of-plane shapes 65 are also readily achievable as needed. It should be appreciated that this ability to change the shape of endotracheal tube 11 in situ as needed can result in a tremendous reduction in the amount of time necessarily to successfully intubate a patient.

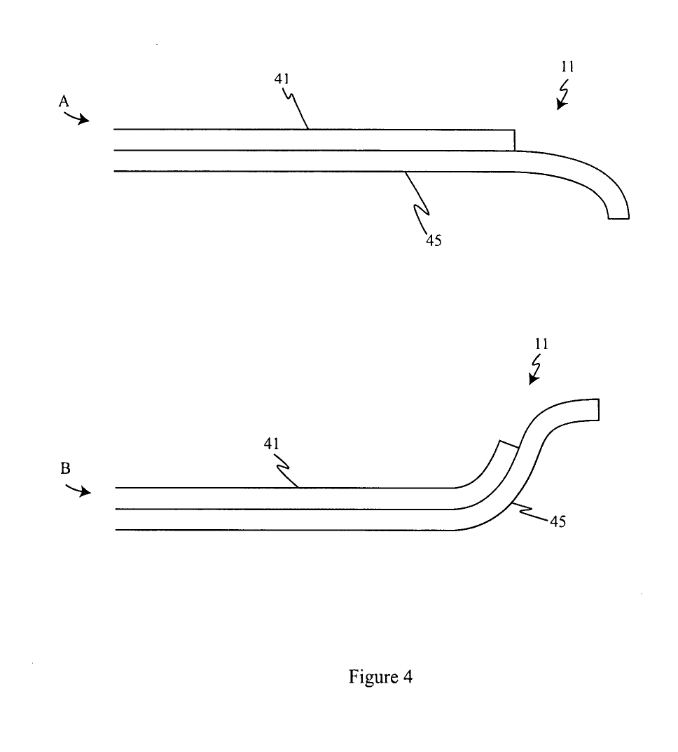

Although endotracheal tube 11 as shown in FIG. 2 has first and second elements 41, 45 coaxial, the present invention is not limited to coaxial elements. As shown in FIG. 4, elements 41 and 45 may be disposed adjacent each other. In FIG. 4A, element 41 is relatively straight, whereas in FIG. 4B element 41 has been curved by the user, causing the overall tube 11 to achieve the shape shown in 4B. Note that there is no requirement that both elements be hollow so long as at least one element has a lumen through which gas may flow.

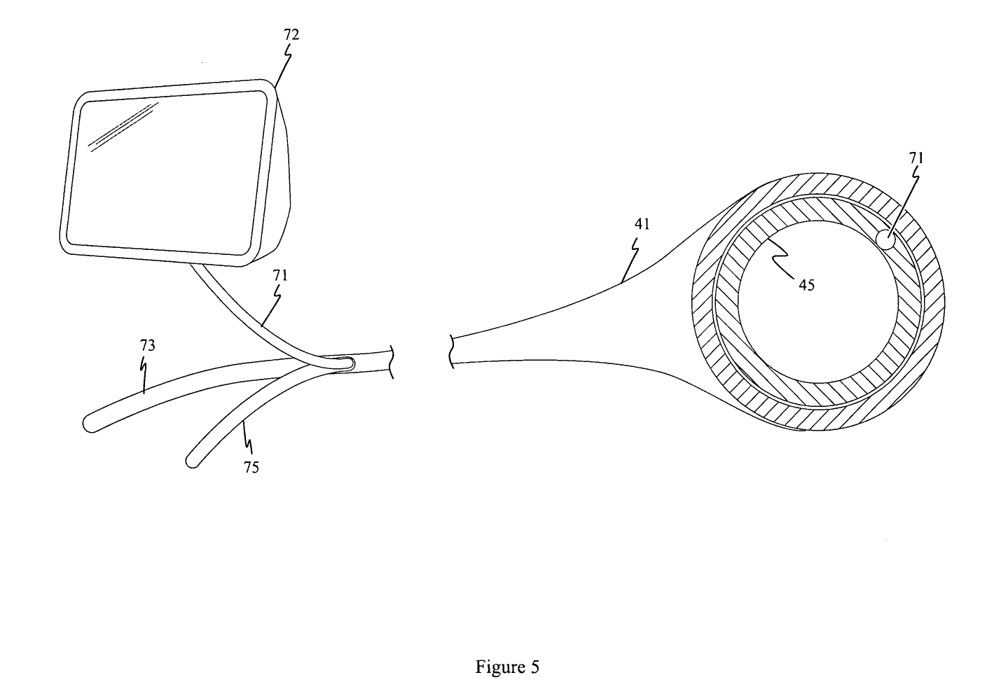

As can be seen in FIG. 5, if desired one of elements 41, 45 can include a fiberscope 71 for displaying to the medical practitioner on a display 72 a view of the airway in the vicinity of the distal end of endotracheal tube 11. Such fiberscopes are sometimes used to facilitate proper intubation in difficult cases. FIG. 5 also shows the pull-wires 73 and 75 by means of which the medical practitioner can independently vary the first and second elements 41, 45 to thereby vary the shape of the endotracheal tube. It should be appreciated that pull-wires, if used, extend from the proximal portion of the endotracheal tube to a convenient position for manipulation by the medical practitioner.

As mentioned above, the endotracheal tube may include a cuff 77 (FIG. 2) which can be inflated in the conventional manner once tube 11 is properly placed. Tube 11 may be an orotracheal tube or a nasotracheal tube. Although tube 11 has been shown and described as a stand-alone endotracheal tube, it should be understood that the same structure may be used as a stylet for a conventional endotracheal tube. Such a stylet is superior to many existing stylets in that it need not be removed from the body to be reshaped. When functioning as a stylet, the overall diameter thereof is preferably less than approximately 8 mm so as to allow the stylet to be disposed in an endotracheal tube. The stylet should also have sufficient rigidity to shape the endotracheal tube in which it is placed. Also preferably the stylet has a length of at least 140 mm for pediatric use and of at least 330 mm for adult use.

In view of the above, it will be seen that the various objects and features of the present invention are achieved and other advantageous results obtained. The description of the invention contained herein is for illustrative purposes only. The invention itself is defined by the claims which follow.

What is claimed is:

1. An endotracheal tube having more than six degrees of freedom.

2. The endotracheal tube as set forth in claim 1 wherein said tube includes a first element having a distal end portion which is capable of achieving a curved configuration and a second element having a distal end portion which is capable of achieving a curved configuration, said first and second elements being rotationally and translationally movable with respect to each other.

3. The endotracheal tube as set forth in claim 2 wherein the shape of the tube may be varied by a user while the tube is at least partially inserted in a living body.

4. The endotracheal tube as set forth in claim 2 wherein the shape of the tube may be varied as desired by a user to accommodate variations in the endotracheal pathway of a living body in which the tube is being inserted.

5. The endotracheal tube as set forth in claim 2 wherein the first and second elements are coaxial.

6. The endotracheal tube as set forth in claim 2 wherein the first and second elements are not coaxial.

7. The endotracheal tube as set forth in claim 2 wherein at least one of the elements includes a fiberscope.

8. The endotracheal tube as set forth in claim 2 wherein the first and second elements are controllable independently by a user to vary the shape of the endotracheal tube.

9. The endotracheal tube as set forth in claim 2 wherein at least one of said elements includes a cuff for inflation once the tube is properly placed.

10. The endotracheal tube as set forth in claim 1 wherein the tube is an orotracheal tube.

11. The endotracheal tube as set forth in claim 1 wherein the tube is a nasotracheal tube.

12. A stylet for an endotracheal tube comprising:
a first element having a distal end portion capable of achieving a curved configuration;
a second element having a distal end portion capable of achieving a curved configuration;
said first and second elements being disposed with respect to each other to form a stylet;
said stylet having a diameter of less than approximately 8 mm so as to be disposed in an endotracheal tube and having sufficient rigidity to shape the endotracheal tube in which it is placed.

13. The stylet as set forth in claim 12 wherein said stylet has a length of at least 140 mm.

14. The stylet as set forth in claim 13 wherein said stylet has a length of at least 330 mm.
